(19) United States
(12) Patent Application Publication
(10) Pub. No.: US 2017/0350893 A1
(43) Pub. Date: Dec. 7, 2017

(54) Biomarker And Therapy Intervention For Malignancy Risk Patients

(71) Applicant: Oxford University Innovation Limited, Oxford (GB)

(72) Inventors: Paul Neil HARDEN, Oxford (Oxfordshire) (GB); Kathyrn Jayne WOOD, Headington Oxford (Oxfordshire) (GB); Matthew James BOTTOMLEY, Headington Oxford (Oxfordshire) (GB)

(21) Appl. No.: 15/521,091

(22) PCT Filed: Oct. 23, 2015
(86) PCT No.: PCT/GB2015/053176

§ 371 Date: Apr. 21, 2017

(30) Foreign Application Priority Data
Oct. 23, 2014 (GB) 1418896.5

(51) Int. Cl. G01N 33/574

(52) U.S. Cl. CPC G01N 33/5743; G01N 2800/50; G01N 2333/70596; G01N 2333/70514; G01N 2333/70517; G01N 2333/70521

(57) ABSTRACT

The invention relates to a method of determining an increased risk of cancer in an immunosuppressed patient, the method comprising: (a) determining the percentage of CD8+CD57+ T-cells in a population of CD8+ T-cells in a sample from the patient; wherein a percentage of 40% or greater of CD8+CD57+ T-cells is indicative of an increased risk of cancer; and/or (b) determining the percentage of CD4+CD57+ T-cells in a population of CD4+ T-cells in a sample from the patient; wherein a percentage of 10% or greater of CD4+CD57+ T-cells is indicative of an increased risk of cancer.

### CROSS-REFERENCE TO RELATED APPLICATION

This application is the National Stage of International Application No. PCT/GB2015/053176, filed 23 Oct. 2015, which claims the benefit of and priority to GB Application No. 1418896.5, having the title “Biomarker And Therapy Intervention For Malignancy Risk Patients,” filed on 23 Oct. 2014, the entire disclosures of which are incorporated by reference in their entireties as if fully set forth herein.

### TECHNICAL FIELD

This invention relates to a method for determining an increased risk of cancer in immunosuppressed patients, use of a cell marker, and kits for enabling the determination of increased risk of cancer.

### BACKGROUND

Transplantation has brought improved survival and quality of life to patients with end-stage renal disease. There has been a steady improvement in short-term graft survival over the last 20 years, due to more refined tissue typing and reduced incidence of acute rejection, surgical complications and opportunistic infections in the post-transplant period. One-year graft survival in both the US and the UK in 2010 was in excess of 94%, compared to 87% and 95% respectively in 1991[1, 2]. One-year patient survival was consistently over 96% during this time. However, long-term patient survival rates in the USA during this period appear to have plateaued or even decreased[2]. Following transplantation, recipients are required to take life-long medication (“immunosuppression”) to prevent the immune system from attacking the graft.

Half of all ‘graft’ loss at 10 years post-transplant is due to death with a functioning graft (DWFG). Whilst the burden of cardiovascular disease and infection are falling, death due to malignancy is climbing, and may have now replaced cardiovascular disease as the leading cause of DWFG[3]. The increasing incidence of malignancy is likely due to interplay of multiple factors, such as an increasing average recipient age at transplantation, increasing long-term graft survival and more potent immunosuppression. Immunosuppression impacts upon graft and patient survival both directly (e.g. graft toxicity) and indirectly (e.g. dyslipidaemia, carcinogenicity and increased susceptibility to infection).

Malignancy is at least three times more common in transplant recipients compared to the general population. Cutaneous malignancy represents the majority of cancer seen long-term post-transplant, of which over 95% is non-melanoma skin cancer (NMSC). Over 80% of NMSC is squamous cell carcinoma (SCC), with the vast majority of the rest being basal cell carcinoma (BCC), which rarely metastasises. Cumulative incidence ranges from 40% in the UK to over 80% in Australia by 20 years after transplantation, representing a 65- to 200-fold increased risk compared with the general population (FIG. 1). This geographical variation (with increased incidence with decreasing proximity to the equator) supports the role for UV radiation and sunburn as risk factors for NMSC, as in the general population.

In the UK, the mean time to first NMSC is eight years post-transplant. NMSC in transplant recipients occurs at a younger age and is more aggressive, with a 5-8% rate of SCC metastasis (compared to less than 5% in the general population). Previous SCC is a predictor for the development of further multifocal NMSC; up to 88% of Western European renal transplant recipients (RTR) may develop a second tumour within 5 years, with a median interval of 10 months[4]. Current UK guidelines recommend that RTR are taught to self-examine for dermatological lesions on a regular basis, and should be examined on an annual basis by a trained healthcare professional[5]. The provision of dermatological screening to all transplant recipients represents a considerable financial outlay for healthcare providers, and is likely to increase as the number of long-term RTR increases (179,361 RTR in the US in 2010[6]). To reduce this burden, attempts have been made to guide the identification of patients at increased risk of NMSC through risk scores based on clinical features (such as age at transplant, skin type, previous sunburn etc.), in order to allow for more targeted monitoring[?-9]. However, these risk scores have not been validated prospectively and are not generally in clinical use.

Following the development of NMSC, in particular SCC, current guidelines recommend that overall levels of immunosuppression should be reduced[5]. However, this is not universally applied and often requires the development of multiple NMSC before immunosuppression reduction is considered. One of the key barriers is the fear of transplant rejection, whereby the immune system attacks and damages the graft, due to over-reduction of immunosuppression. Pro-active reduction of immunosuppression is associated with reduced incidence of malignancy long term[10]. There are no objective markers to guide the optimal degree of immunosuppression on an individual basis; thus in stable long-term RTR dosage adjustments are usually reactive, in response to clinical events (such as malignancy), by which time the opportunity to prevent the complications of over-immunosuppression has been missed.

Previous work undertaken in RTR has found that alterations in regulatory cell populations in peripheral blood may be predictive of future SCC risk, but this method of stratification appears to only have short-term predictive value (up to 100 days)[11-13]. However, other studies have found that effector populations within SCC from RTR show an altered phenotype, such as reduced CD8+ T-cell infiltration and altered cytokine profile compared to non-RTR[11, 14-16].

### SUMMARY

An aim of the present invention is to provide an improved surveillance or therapy regime for immunosuppressed patients at risk of cancer.

According to a first aspect of the invention, there is provided a method of determining an increased risk of cancer in an immunosuppressed patient, the method comprising:
(a) determining the percentage of CD8+CD57+ T-cells in a population of CD8+ T-cells in a sample from the patient;
wherein a percentage of 40% or greater of CD8+CD57+ T-cells is indicative of an increased risk of cancer; and/or
(b) determining the percentage of CD4+CD57+ T-cells in a population of CD4+ T-cells in a sample from the patient;
wherein a percentage of 10% or greater of CD4+CD57+ T-cells is indicative of an increased risk of cancer.

In one embodiment, the method may comprise:
determining the percentage of CD8+CD57+ T-cells in a population of CD8+ T-cells in a sample from the patient;
   wherein a percentage of 40% or greater of CD8+CD57+ T-cells is indicative of an increased risk of cancer.

In one embodiment, the method may comprise:
determining the percentage of CD4+CD57+ T-cells in a population of CD4+ T-cells in a sample from the patient;
wherein a percentage of 10% or greater of CD4+CD57+ T-cells is indicative of an increased risk of cancer.

The invention advantageously provides CD57 as a new and accurate biomarker for risk assessment and factor in treatment choice of immunosuppressed patients. CD57 also beneficially acts as positive marker (e.g. it is confirmed by its presence, in contrast to a negative marker, which is confirmed by its absence). CD57 (HNK-1, Leu-7) is a terminally sulphated carbohydrate epitope found on various cell surface glycoproteins on T- and NK-cells, as well as in the central nervous system[17]. The function of CD57 specifically is unclear; it has been found on the gp130 subunit of the IL-6 receptor in resting lymphocytes[18], whilst in the nervous system it is mostly expressed on adhesion proteins[19]. CD57 is thought to represent a marker of terminal differentiation and functional exhaustion in lymphocytes, as CD57⁺ T-cells possess shortened telomeres, low expression of cell cycle proteins, and may (debatably) have limited proliferative capacity[20, 21]. CD57 expression is thought to be induced through chronic antigenic stimulation, and (in support of this) increased numbers of CD57-expressing T-cells are found in both solid-organ and haematopoietic transplantation and chronic viral infection [22-31]. Given the role of CD8+ T-cells in detecting and eliminating early transformed and virally infected cells, it was found herein that a deficit in this, as manifested by CD57 expression, has predictive power for development of malignancy, such as SCC. In particular, the excess risk in patients with a majority of CD8 cells expressing this marker (>50%—‘CD57hi’) is 4 times that of those with a low number (<50%—‘CD57lo’) expressing this marker, after correction for age and previous SCC. This represents one of the strongest markers of subsequent SCC development identified.

Furthermore, this marker can advantageously represent a way of stratifying long-term risk of cancer at a much earlier timepoint, and therefore can be used in longitudinal studies.

In one embodiment, a percentage of 45% or greater of CD8+CD57+ T-cells is indicative of an increased risk of cancer. In another embodiment, a percentage of 50% or greater of CD8+CD57+ T-cells is indicative of an increased risk of cancer. In another embodiment, a percentage of 55% or greater of CD8+CD57+ T-cells is indicative of an increased risk of cancer. In another embodiment, a percentage of 57% or greater of CD8+CD57+ T-cells is indicative of an increased risk of cancer. In another embodiment, a percentage of 60% or greater of CD8+CD57+ T-cells is indicative of an increased risk of cancer. Alternatively, a percentage of 65% or greater of CD8+CD57+ T-cells is indicative of an increased risk of cancer.

The CD57+ T-cells may also be CD3+CD8+CD4− T-cells. Additionally, or alternatively the CD57+ T-cells may also be CD3+CD4+CD8− T-cells.

In one embodiment, a percentage of 11% or greater of CD4+CD57+ T-cells is indicative of an increased risk of cancer. In another embodiment, a percentage of 12% or greater of CD4+CD57+ T-cells is indicative of an increased risk of cancer. In another embodiment, a percentage of 13% or greater of CD4+CD57+ T-cells is indicative of an increased risk of cancer. In another embodiment, a percentage of 14% or greater of CD4+CD57+ T-cells is indicative of an increased risk of cancer. In another embodiment, a percentage of 15% or greater of CD4+CD57+ T-cells is indicative of an increased risk of cancer. In another embodiment, a percentage of 18% or greater of CD4+CD57+ T-cells is indicative of an increased risk of cancer.

Advantageously, it was determined that patients with a high proportion of CD3+CD8+CD4− T-cells expressing CD57 also have an increased number of CD3+CD4+CD8− T-cells (another type of T-cell) expressing CD57, and this can also have predictive value in the risk for cancer.

The method may further comprise the step of selecting patients determined to be at increased risk of cancer for one or more of:
increased surveillance for cancer;
modifying the immunosuppressive therapy regime; or
providing preventative therapy for cancer, and optionally wherein the preventative therapy for cancer is anti-cancer therapy.

Additionally or alternatively to determining the percentage of CD57+ T-cells, the percentage of CD28− T-cells may be determined, wherein a percentage of 40% or greater of CD28− T-cells is indicative of an increased risk of cancer. Additionally or alternatively to determining the percentage of CD57+ T-cells, the percentage of CD28− T-cells may be determined, wherein a percentage of 45% or greater of CD28− T-cells is indicative of an increased risk of cancer. Additionally or alternatively to determining the percentage of CD57+ T-cells, the percentage of CD28− T-cells may be determined, wherein a percentage of 50% or greater of CD28− T-cells is indicative of an increased risk of cancer. Additionally or alternatively to determining the percentage of CD57+ T-cells, the percentage of CD28− T-cells may be determined, wherein a percentage of 55% or greater of CD28− T-cells is indicative of an increased risk of cancer. Additionally or alternatively to determining the percentage of CD57+ T-cells, the percentage of CD28− T-cells may be determined, wherein a percentage of 60% or greater of CD28− T-cells is indicative of an increased risk of cancer.

According to another aspect of the invention, there is provided a method of determining an increased risk of cancer in an immunosuppressed patient, the method comprising:
determining the percentage of CD8+CD28− T-cells in a sample of CD8+ T-cells from the patient;
   wherein a percentage of 40% or greater is indicative of an increased risk of cancer.

The percentage of CD8+CD28− T-cells may be determined, wherein a percentage of 45% or greater of CD8+CD28− T-cells in a population of CD8+ T-cells is indicative of an increased risk of cancer. The percentage of CD8+CD28− T-cells may be determined, wherein a percentage of 50% or greater of CD8+CD28− T-cells in a population of CD8+ T-cells is indicative of an increased risk of cancer. The percentage of CD8+CD28− T-cells may be determined, wherein a percentage of 55% or greater of CD8+CD28− T-cells in a population of CD8+ T-cells is indicative of an increased risk of cancer. The percentage of CD8+CD28− T-cells may be determined, wherein a percentage of 60% or greater of CD8+CD28− T-cells in a population of CD8+ T-cells is indicative of an increased risk of cancer.

In addition to determining the percentage of CD8+CD28− T-cells, the method may comprise determining the percentage of CD4+CD57+ T-cells in a population of CD4+ T-cells in a sample from the patient;
wherein a percentage of 10% or greater of CD4+CD57+ T-cells is indicative of an increased risk of cancer.

The invention may provide the use of CD57 and/or CD28 expression to stratify patients, in particular RTR patients, and identify those at an increased risk of NMSC, and in particular SCC. Preferably an increased risk is a risk that is at least 2 fold, 3 fold, 4 fold or more, higher than that observed in the general population.

In one embodiment, the method may comprise the step of selecting patients determined to be at increased risk of cancer for increased surveillance for cancer. In other embodiments the method may comprise the step of selecting patients determined to be at increased risk of cancer for modifying the immunosuppressive therapy regime. In another embodiment the method may comprise the step of selecting patients determined to be at increased risk of cancer for providing preventative therapy for cancer. In a further embodiment the method may comprise the step of selecting patients determined to be at increased risk of cancer for providing anti-cancer therapy. Combinations of these embodiments may be provided, such as selection for increased surveillance for cancer and selection for modifying the immunosuppressive therapy regime.

For selected patients determined to be at increased risk of cancer the method may further comprise one or more steps comprising:
increasing surveillance for cancer;
modifying the immunosuppressive therapy regime; or
providing preventative therapy for cancer, and optionally wherein the preventative therapy for cancer is anti-cancer therapy.

In one embodiment, the method may comprise the step of increasing surveillance for cancer in selected patients determined to be at increased risk of cancer. In other embodiments the method may comprise the step of modifying the immunosuppressive therapy regime in selected patients determined to be at increased risk of cancer. In another embodiment the method may comprise the step of providing preventative therapy for cancer for selected patients determined to be at increased risk of cancer. In a further embodiment the method may comprise the step of providing anti-cancer therapy for selected patients determined to be at increased risk of cancer. Combinations of these embodiments may be provided, such as increasing surveillance for cancer and modifying the immunosuppressive therapy regime for selected patients determined to be at increased risk of cancer.

Immunosuppression reduction for reduced cancer risk is a benefit of the invention. Immunosuppression also has adverse effects on other patient outcomes, such as cardiovascular risk (through induction of elevated lipids in blood, diabetes, etc), as well as other adverse effects (such as steroid use, which can lead to cataracts). Therefore, reducing immunosuppression in accordance with the invention can have a beneficial effect on these complications, further benefiting the patient, and reducing the burden on healthcare resources.

Modifying the immunosuppressive therapy regime may comprise one or more of:
reduction of dose and/or frequency of immunosuppressive therapy;
switching one or more immunosuppressive drugs to an alternative immunosuppressive drug(s);
reducing the number of different immunosuppressive drugs administered to the patient.

In one embodiment, modifying the immunosuppressive therapy regime may comprise reduction of dose of immunosuppressive therapy. Additionally or alternatively, modifying the immunosuppressive therapy regime may comprise reduction of frequency of immunosuppressive therapy. In one embodiment, modifying the immunosuppressive therapy regime may comprise reduction of dose of immunosuppressive therapy by at least 10% reduction. In another embodiment, modifying the immunosuppressive therapy regime may comprise reduction of dose of immunosuppressive therapy by at least 15% reduction. In another embodiment, modifying the immunosuppressive therapy regime may comprise reduction of dose of immunosuppressive therapy by at least 20% reduction. Modifying the immunosuppressive therapy regime may comprise reduction of dose of immunosuppressive therapy by at least 25% reduction. Modifying the immunosuppressive therapy regime may comprise reduction of dose of immunosuppressive therapy by at least 30% reduction. Modifying the immunosuppressive therapy regime may comprise reduction of dose of immunosuppressive therapy by at least 35% reduction. Modifying the immunosuppressive therapy regime may comprise reduction of dose of immunosuppressive therapy by at least 40% reduction. Modifying the immunosuppressive therapy regime may comprise reduction of dose of immunosuppressive therapy by at least 45% reduction. Modifying the immunosuppressive therapy regime may comprise reduction of dose of immunosuppressive therapy by at least 50% reduction. Modifying the immunosuppressive therapy regime may comprise reduction of dose of immunosuppressive therapy by at least 55% reduction. Modifying the immunosuppressive therapy regime may comprise reduction of dose of immunosuppressive therapy by at least 60% reduction. Modifying the immunosuppressive therapy regime may comprise reduction of dose of immunosuppressive therapy by between about 5% and 60% reduction. Modifying the immunosuppressive therapy regime may comprise reduction of dose of immunosuppressive therapy by between about 10% and 60% reduction. Modifying the immunosuppressive therapy regime may comprise reduction of dose of immunosuppressive therapy by between about 10% and 60% reduction. Modifying the immunosuppressive therapy regime may comprise reduction of dose of immunosuppressive therapy by between about 10% and 50% reduction. Modifying the immunosuppressive therapy regime may comprise reduction of dose of immunosuppressive therapy by between about 20% and 50% reduction.

Modifying the immunosuppressive therapy regime may comprise reduction of frequency of immunosuppressive therapy from daily to every other day. Modifying the immunosuppressive therapy regime may comprise reduction of frequency of immunosuppressive therapy from twice daily to once daily.

Additionally or alternatively, modifying the immunosuppressive therapy regime may comprise reduction of the frequency of immunosuppressive therapy (e.g. less frequent dosage).

In another embodiment, modifying the immunosuppressive therapy regime may comprise switching one or more immunosuppressive drugs to an alternative immunosuppressive drug(s). The skilled person will be aware of alternative drugs, and alternative combinations of drugs, available for immunosuppression which may provide a different mechanism of action or effect, for example for prevention of transplant rejection.

In another embodiment, modifying the immunosuppressive therapy regime may comprise reducing the number of different immunosuppressive drugs administered to the patient. For example, where immunosuppressive therapy comprises administration of a first drug, such as MMF, and a second drug, such as tacrolimus, the immunosuppressive therapy regime may be modified to just the first drug (e.g. MMF) or just the second drug (e.g. tacrolimus).

The patient at increased risk of cancer may be selected for both a modification of the immunosuppressive therapy regime and an increased surveillance for cancer.

Modification of the immunosuppressive therapy regime may be accompanied by an increase in surveillance of transplant rejection. For example renal transplant rejection indicators may be monitored, or monitored more frequently for patients receiving a reduced immunosuppressive therapy.

The method may be for a long term prognosis. For example, the percentage of 50% or greater may be indicative of a risk of cancer after a period of at least 5 years. The percentage of 50% or greater may be indicative of a risk of cancer after a period of at least 6 years, 8 years, 9 years, 10 years, or 15 years, following the onset of immunosuppression in the patient. For example, the percentage of 40% or greater may be indicative of a risk of cancer after a period of at least 5 years. The percentage of 40% or greater may be indicative of a risk of cancer after a period of at least 6 years, 8 years, 9 years, 10 years, or 15 years, following the onset of immunosuppression in the patient. For example, the percentage of 45% or greater may be indicative of a risk of cancer after a period of at least 5 years. The percentage of 45% or greater may be indicative of a risk of cancer after a period of at least 6 years, 8 years, 9 years, 10 years, or 15 years, following the onset of immunosuppression in the patient.

Increased surveillance may be a more frequent and/or more detailed and investigative cancer check-up. The check-up may be a medical examination and/or use of a diagnostic kit or device capable of identifying cancer or pre-cancer irregularities.

The immunosuppression may be due to the patient receiving immunosuppressive therapy. Alternatively, the immunosuppression may be as a result of a disease, such as an infection or chronic condition which suppressed the immune system. The immunosuppression may be as a result of inflammatory bowel disease, asthma or autoimmune diseases such as rheumatoid arthritis and vasculitis (such as SLE) which require long-term, ongoing immunosuppression. The immunosuppression may be as a result of a side-effect from a drug regime. The immunosuppression may be as a result of radiation therapy or chemotherapy.

The immunosuppressive therapy may comprise a regular dosage of one or more immunosuppressant drug(s). The immunosuppressive therapy may comprise a regular dosage of combinations of immunosuppressant drugs.

An immunosuppressant drug may comprise any drug selected from the group comprising a calcineurin inhibitor, such as tacrolimus (also known as FK-506 or fujimycin, trade names Prograf™, Advagraf™, Protopic™), pimecrolimus, ciclosporin (Novartis' Sandimmune™, Neoral™) or sirolimus (rapamycin, trade name Rapamune™); azathioprine; mycophenolate mofetil (MMF, trade name CellCept™); mycophenolic acid (MPA), or Myfortic™, cyclophosphamide; belatacept; and steroids, such as corticosteroids (e.g. prednisolone) or glucocorticoids; or combinations thereof. The skilled person will understand that the immunosuppressant drug may be any current or yet to be licensed or discovered molecule that has the ability to suppress the immune system of a patient.

The immunosuppressive therapy may not comprise a regular and ongoing dosage of steroid. The immunosuppressive therapy following kidney transplantation may not comprise a regular and ongoing dosage of steroid.

The immunosuppressive therapy (i.e. prior to any modification/reduction according to the invention), for example for a typical kidney transplant patient, may comprise azathioprine at about 1.5 mg/kg daily or mycophenolate mofetil (MMF) at about 750 mg twice daily; and tacrolimus (or ciclosporin) at about 0.05 mg/kg twice daily aiming for trough levels of 8-12 ng/ml for 6 months, then 5-10 ng/ml long-term.

The immunosuppressive therapy (i.e. prior to any modification/reduction according to the invention), for example for a typical lung transplant patient, may comprise steroids at 0.5-1 mg/kg daily (e.g. 30-60 mg per day for an average 60 kg person), aiming to wean over a number of months to 5-10 mg per day for an average 60 kg person (long-term); and azathioprine 1-2 mg/kg daily or MMF 1000-1500 mg twice daily; and tacrolimus at 0.05 mg/kg twice daily aiming for trough levels of 8-15 ng/ml.

The immunosuppressive therapy (i.e. prior to any modification/reduction according to the invention) may comprise azathioprine at least 1.5 mg/kg daily or MMF at least 750 mg twice daily; and optionally tacrolimus (or ciclosporin) at least 0.05 mg/kg twice daily aiming for trough levels of at least 5 ng/ml.

The immunosuppressive therapy (i.e. prior to any modification/reduction according to the invention) may comprise azathioprine at least 1.1 mg/kg daily or MMF at least 1010 mg twice daily; and optionally tacrolimus (or ciclosporin) at least 0.05 mg/kg twice daily aiming for trough levels of at least 9 ng/ml. The immunosuppressive therapy (i.e. prior to any modification/reduction according to the invention) may comprise azathioprine at least 1.1 mg/kg daily or MMF at least 1000 mg twice daily; and optionally tacrolimus (or ciclosporin) at least 0.05 mg/kg twice daily aiming for trough levels of at least 9 ng/ml. The immunosuppressive therapy (i.e. prior to any modification/reduction according to the invention) may comprise azathioprine at least 1.1 mg/kg daily or MMF at least 1250 mg twice daily; and optionally tacrolimus (or ciclosporin) at least 0.05 mg/kg twice daily aiming for trough levels of at least 9 ng/ml.

The immunosuppressive therapy (i.e. prior to any modification/reduction according to the invention) may comprise azathioprine at least 1.5 mg/kg daily or MMF at least 1100 mg twice daily; and optionally tacrolimus (or ciclosporin) at least 0.05 mg/kg twice daily aiming for trough levels of at least 8.1 ng/ml. The immunosuppressive therapy (i.e. prior to any modification/reduction according to the invention) may comprise azathioprine at least 1.5 mg/kg daily or MMF at least 1000 mg twice daily; and optionally tacrolimus (or ciclosporin) at least 0.05 mg/kg twice daily aiming for trough levels of at least 8.1 ng/ml. The immunosuppressive therapy (i.e. prior to any modification/reduction according to the invention) may comprise azathioprine at least 1.5 mg/kg daily or MMF at least 1250 mg twice daily; and optionally tacrolimus (or ciclosporin) at least 0.05 mg/kg twice daily aiming for trough levels of at least 8.1 ng/ml.

Where azathioprine is administered for immunosuppressive therapy (i.e. prior to any modification/reduction according to the invention) at least 1 mg/kg may be administered daily, or at least 1.1 mg/kg may be administered daily, or at least 1.5 mg/kg may be administered daily, or at least 25 mg/kg may be administered daily.

Where MMF is administered for immunosuppressive therapy (i.e. prior to any modification/reduction according to the invention) at least 750 mg may be administered twice daily, or at least 900 mg may be administered twice daily, or at least 1000 mg may be administered twice daily, or at least 1010 mg may be administered twice daily, or at least 1100 mg may be administered twice daily, or at least 1250 mg may be administered twice daily, or at least 1500 mg may be administered twice daily.

Where tacrolimus (or ciclosporin) is administered for immunosuppressive therapy (i.e. prior to any modification/reduction according to the invention) at least about 0.05 mg/kg may be administered twice daily aiming for trough levels of at least 8 ng/ml for 6 months, then at least 5 ng/ml long-term. Where tacrolimus (or ciclosporin) is administered for immunosuppressive therapy (i.e. prior to any modification/reduction according to the invention) at least about 0.05 mg/kg may be administered twice daily aiming for trough levels of at least 10 ng/ml for 6 months, then at least 8 ng/ml long-term. Where tacrolimus (or ciclosporin) is administered for immunosuppressive therapy (i.e. prior to any modification/reduction according to the invention) at least about 0.05 mg/kg may be administered twice daily aiming for trough levels of at least 12 ng/ml for 6 months, then at least 10 ng/ml long-term.

The immunosuppressive therapy may additionally comprise ongoing administration of steroid. The immunosuppressive therapy may additionally comprise administration of steroid at least 5-10 mg per day for an average 60 kg person, or at least 5 mg per day for an average 60 kg person, or at least 8 mg per day for an average 60 kg person, or at least 10 mg per day for an average 60 kg person.

The immunosuppressive therapy may be following a transplant. The transplant may comprise a kidney transplant. The transplant may comprise a heart and/or lung transplant. The transplant may not be a liver transplant.

The cancer may be skin cancer. The skin cancer may be SCC (squamous cell carcinoma). The SCC may be following a transplant, such as a kidney transplant, and an associated immunosuppressive drug regime.

The patient may be a mammal. The patient may be a human. The patient may be over 30 years old. The patient may be over 35, 40, 45, 50, 55, 60, or 65 years old. The patient may have been immunosuppressed for at least 5 years. The patient may have been immunosuppressed for at least 8, 10, 12, 15, 18, 20, or 25 years. The patient may be a smoker, or ex-smoker. The patient may be pale skinned (as assessed by Fitzpatrick skin type). The patient may be skin type 1-4 (as assessed by Fitzpatrick skin type). The patient may be skin type 1-4 (as assessed by Fitzpatrick skin type) and greater than 55 years old at the time of the transplant/start of immunosupression. The patient may have received above average UV exposure relative to the general population. The patient may have received at least 2-3, or 5 or more severe sunburns in their past history. The patient may have green or blue eyes. The patient may have worked in a profession based outdoors. The patient may have lived in a tropical climate, in which UV exposure was more significant.

The patient may have had a transplant. The patient may be a RTR—having received a kidney transplant. The transplant may comprise a heart and/or lung transplant. The transplant may not be a liver transplant. Preferably, the patient has had a transplant and is having immunosuppressive therapy.

The sample may comprise a blood sample. Alternatively or additionally, the sample may comprise a tissue sample. For example, in patients with tumours, the sample may comprise a tumour biopsy.

The determination of the percentage of CD8+CD57+ or CD8+CD28− T-cells in a population of CD8+ T-cells may comprise the use of flow cytometry and fluorophore-tagged antibodies to identify and quantify the number of cells.

According to another aspect of the invention, there is provided the use of CD57 as a biomarker for determining an increased risk of cancer in a patient receiving immunosuppressive therapy.

According to another aspect of the invention, there is provided the use of CD28 as a biomarker for determining an increased risk of cancer in a patient receiving immunosuppressive therapy.

An increased risk of cancer may be defined as an at least 2 fold, 3 fold, 4 fold or more, increase in the risk of getting cancer.

The immunosuppressive therapy may be following a transplant. The transplant may comprise a kidney transplant. The cancer may comprise skin cancer, such as SCC.

According to another aspect of the invention, there is provided the use of CD28 as a biomarker for determining an increased risk of cancer in a patient receiving immunosuppressive therapy following a kidney transplant.

In one embodiment, the patient may be pre-screened for CD57 percentage prior to receiving immunosuppressive therapy (e.g. before a transplant), in order to help to determine the level of immunosuppressant therapy appropriate to reduce the risk of cancer.

According to another aspect of the invention, there is provided a kit for determining an increased risk of cancer in a patient receiving immunosuppressive therapy comprising:
a CD8 binding agent
a CD57 binding agent and/or a CD28 binding agent.

The kit may further comprise a CD4 binding agent.

According to another aspect of the invention, there is provided a kit for determining an increased risk of cancer in a patient receiving immunosuppressive therapy comprising:
a CD4 binding agent
a CD57 binding agent and/or a CD28 binding agent.

The kit may further comprise a CD8 binding agent.

The kit may further comprise guidance on the percentage threshold of CD57+ and/or CD28− T-cells in a population of CD8+ T-cells, wherein the guidance advises on the modification of an immunosuppression regime of a patient being tested. Additionally, or alternatively the kit may further comprise guidance on the percentage threshold of CD57+ and/or CD28− T-cells in a population of CD4+ T-cells, wherein the guidance advises on the modification of an immunosuppression regime of a patient being tested.

The percentage threshold of CD57+ and/or CD28− T-cells in a population of CD8+ T-cells in the kit may be in accordance with the invention herein (e.g. 40% or greater, or 45% or greater, or 50% or greater, e.t.c.).

The binding agent(s) may comprise a labelled antibody, antibody fragment, or mimic thereof. The label may comprise a fluorophore. Each binding agent for respective CD markers may comprise a different fluorophore to enable differentiation therebetween.

The term “immunosuppression” may be defined as the use of exogeneous compounds on an ongoing basis to impair a potentially pathogenic immune response. The immunosuppression may require systemic administration of the exogeneous compounds.

The skilled person will understand that optional features of one embodiment or aspect of the invention may be applicable, where appropriate, to other embodiments or aspects of the invention.

### BRIEF DESCRIPTION OF THE DRAWINGS

Embodiments of the invention will now be described in more detail, by way of example only, with reference to the accompanying drawings.

FIG. 1: Cumulative incidence of non-melanoma skin cancer in Australia (left) and the UK (right—dashed line represents all NMSC whilst solid line represents SCC).

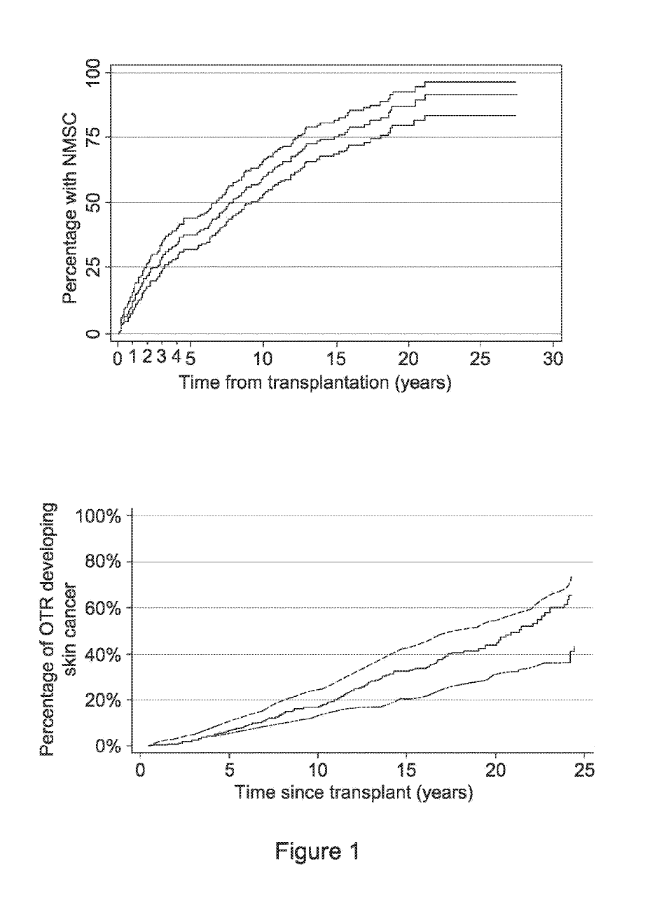

FIG. 2: Kaplan-Meier survival graph illustrating time from enrolment to SCC development, stratified by history of previous SCC.

FIG. 3: FIGS. 3A to 3B, like FIG. 2, show survival graphs illustrating time from enrolment to SCC development, stratified by history of previous SCC. In FIG. 3 the days from enrolment are longer. The data presented further demonstrates that the CD57 phenotype predicts SCC development and recurrence. FIG. 3A shows SCC occurrence in all RTRs by history of previous SCC. FIG. 3B shows SCC occurrence in all RTRs by CD57 phenotype. FIG. 3C shows SCC recurrence in all RTRs by time since previous SCC (for those with SCC during study or preceding year). Black ticks indicate censoring. Hazard ratios are reported as univariate Cox models.

FIG. 4: A) ROC curve for age at enrolment and percentage of CD57+CD8+ cells. RTR was stratified by greater than or less than 50% CD8 cells expressing CD57. B) 2×2 table demonstrating distribution of RTR when stratified by CD57hi or lo. Chi-squared value for the 2×2 table was not significant (p=0.099).

FIG. 5: A) Kaplan-Meier survival curve demonstrating time to SCC in the highest (CD57hi and previous SCC) and lowest risk groups (CD57lo and no previous SCC). B) Kaplan-Meier survival curve for time between SCC in those with a previous SCC, stratified by CD57hi and CD57lo.

FIG. 6: IL-2 production in healthy PBMC (n=3) is impaired in CD57+ cells, irrespective of whether the cell is of a naïve or memory phenotype.

FIG. 7: Shows the proportion of CD8+ T-cells not expressing CD28 (percentage CD8+CD28−) is equally as predictive as (A) and generally correlates strongly with (B) the proportion of CD8+ T-cells expressing CD57 (percentage CD8+CD57+).

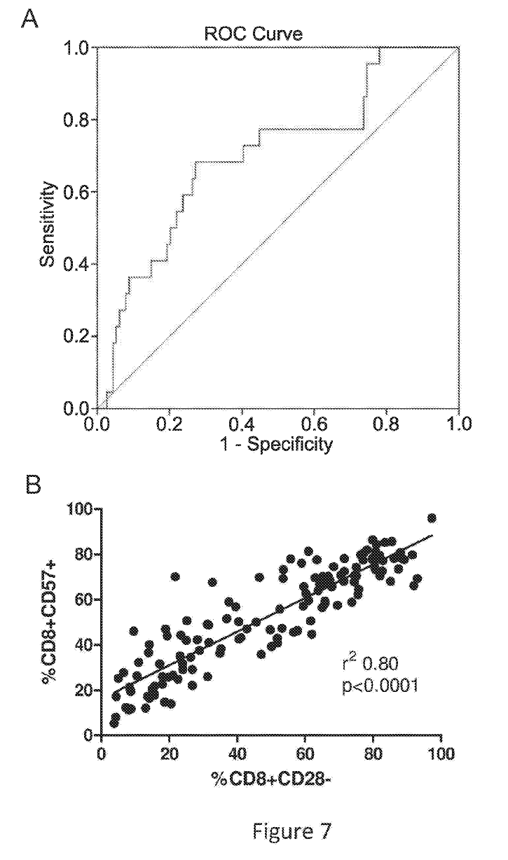

FIG. 8: Demonstrates that CD57 expression upon CD8+ T cells is stable with time and SCC development. Correlation of percentage of CD57-expressing CD8+ T cells between enrolment and 238 days (A) and 385 days (B) later. Points in grey indicate RTRs who developed SCC between sampling. Broken line indicates line of perfect correlation, while solid line indicates line of best fit for whole cohort. Coefficient of determination (r2), standard deviation (SD) and coefficient of variation (CoV) are provided stratified by those who developed SCC between sampling (grey triangles) and those who remained SCC-free (black circles).

### DETAILED DESCRIPTION

### CD57 Expression as a Predictor of Cutaneous Malignancy in RTR

### Introduction

### Methods

### Recruitment

Suitable RTR were approached during routine transplant outpatient follow-up at the Oxford Transplant Centre (or its satellite clinics) and asked to participate in the study. Written consent was provided by all participants. Inclusion criteria are detailed in table 1.

TABLE 1
Recruitment inclusion and exclusion criteria.

| Inclusion Criteria | Exclusion Criteria |
|---|---|
| Male and female RTR aged greater than 18 | Unable/unwilling to provide informed |
| years old. | consent to participate |
| A stable, functioning renal transplant | Previous invasive malignancy during the |
| (defined as serum creatinine increased | last five years (except cutaneous basal cell |
| <30% above previous value in preceding | carcinoma*) |
| 12 weeks). | |
| Have provided informed consent to | Evidence of systemic infection at time of |
| participate | recruitment (see below) |
| | Transplant recipient of any organ other |
| | than kidney previously |
| | Previous evidence of human |
| | immunodeficiency virus (HIV) infection |
| | First cSCC developed within 1 year of first |
| | transplant |

*or cutaneous squamous cell carcinoma in group 1.

RTR with and without a history of prior cutaneous SCC were matched by age, sex and total duration of immunosuppression. A questionnaire was completed regarding sun exposure and risk factors for malignancy development. Renal function was assessed by the serum creatinine performed most recently to time of sampling (in the vast majority this was on the same day) and by calculation of the eGFR (using the four-variable MDRD equation). For the purpose of determining cSCC-free survival, time until occurrence was taken as from the date of recruitment until the date of excision of a histologically-confirmed cSCC. For the purposes of counting discrete cSCC, an event was not counted if it was considered a recurrence; either by description in the medical notes, or report of a scar within the histology (with previous excision at that site within one year). Where there was diagnostic uncertainty (e.g. keratoacanthoma versus cSCC), the reporting histologist's opinion was taken as final. If no opinion was given as to the more likely diagnosis, the event was not included.

### Clinical Risk Scores

Three risk scores for use in transplant patients have been published previously, and all three were calculated for all participants. These were:
Harwood risk score[8] as defined by:

| Risk score | Phenotype |
|---|---|
| 1 | Fitzgerald skin type 5-6; |
| 2 | Fitzgerald skin type 1-4, <35 years old at first |
| | transplant; |
| 3 | Fitzgerald skin type 1-4, 35-44 years old at first |
| | transplant, <5 lifetime sunburns; |
| 4 | As per risk score 3 but >5 lifetime sunburns or 45-54 |
| | years old at first transplant; |
| 5 | Skin type 1-4 and greater than 55 years old at first |
| | transplant. |

Urwin risk score[9] as calculated by the sum of 2 points if age >50 at first transplant; 2 points if average daily lifetime exposure to sunlight >1 hour; 2 points if >30 years in a tropical climate; 3 points if the participant had an SCC prior to transplant; 2 points if the participant had another NMSC prior to transplant; 1 point for any history of childhood sunburn (taken to be <16 years old); 1 point for Fitzgerald skin type 1.
Harden risk score[7] as determined by the risk score (M×1.26)+(A×0.1)+(G×1.87) where M is gender (where male scores 1, female scores 0), A is age at transplantation (in years) and G is eye colour (where green eyes score 1, all other colours score 0).

### Flow Cytometric Analysis

Blood for the study was taken at the same time as routine clinical venepuncture, then immediately stored on ice. All samples were processed within four hours of venepuncture and was anticoagulated with EDTA in vacutainers (BD Biosciences, Oxford, UK).

Peripheral blood mononuclear cells (PBMC) were separated using density-gradient centrifugation with Lymphocyte Separation Medium (GE Healthcare, Amersham, UK). Fresh PBMC were then incubated with a cocktail of monoclonal antibodies including CD3, CD57, CD28 (eBiosciences, Hatfield, UK), CD8 (BD Biosciences, UK) and CD4 (Beckman Coulter, UK) for 45 minutes at 4° C. The stained cells were then analysed using the Navios flow cytometry system (Beckman Coulter).

### Data Analysis

Flow cytometry analysis was performed using Kaluza version 1.2 (Beckman Coulter, UK). Statistical analysis was performed using SPSS 20 (IBM Corp, NY) or Graphpad Prism for Windows version 5.03 (Graphpad, San Diego, Calif.). Results are shown as median (interquartile range) unless specified otherwise, except hazard and odds ratios which are reported as hazard/odds ratio (95% confidence interval). Comparison between groups was performed using the non-parametric two-tailed Mann-Whitney (two groups) or Kruskal-Wallis (multiple groups) test, with continuous variables. For categorical variables the chi-squared or Fisher's exact test were used as appropriate. Where the Kruskal-Wallis test was performed, a subsequent post-hoc Dunn test was applied. Correlations were tested using Pearson's test. Multivariate Cox regression was performed using a backward stepwise method. A p-value of less than 0.05 was considered significant. The statistics presented in this document represent those performed by the author with review by Cristian Ciria and Sharon Love of the Centre for Statistics in Medicine, University of Oxford.

The study received a favourable ethical opinion from the NHS Research Ethics Committee (reference: 12/WS/0288).

### Results (NB Data Collection is Ongoing—Results Accurate as of 13 Oct. 2014)

### Recruitment and Demographics

116 RTR have been recruited to date and the demographics of the group are summarised in Table 2. Of note, those with a history of SCC are older and with a trend towards a lower BMI than those without a history of SCC. Amongst risk factors for SCC, those with a history of SCC were significantly more likely to have a first-degree family history of malignancy, and had a significantly increased Urwin risk score compared to those without a history of SCC (table 2). Retrospectively, RTR with a history of SCC had a trend towards an increased proportion of CD8+ cells expressing CD57; there was no difference in absolute number.

TABLE 2
study participant demographics & clinical phenotype. A smoker was defined as
a participant with >1 pack-year history of smoking. Family history of malignancy was
defined as malignancy in a sibling or parent. Chronic UV exposure was defined as by
employment in an outdoor occupation for greater than five years; continuous residence in
a sunny climate for greater than six months; or more than 11 holidays to a sunny climate
where the participant sunbathed[8].

| | SCC | No SCC | p |
|---|---|---|---|
| Number | 59 | 57 | |
| % male | 71% | 67% | 0.60 |
| % CMV seropositive at enrolment | 68% | 63% | 0.55 |
| Median (IQR) age (yr) at enrolment | 66 | 61 | 0.02 |
| | (58-74) | (55-67) | |
| Median (IQR) age (yr) at 1st Tx | 43 | 40 | 0.26 |
| | (31-52) | (32-47) | |
| Median (IQR) body mass index (kg/m²) at | 25.2 | 26.1 | 0.08 |
| enrolment | (21.7-28.2) | (23.3-29.7) | |
| Median (IQR) i'suppression time (mo) at | 283 | 249 | 0.16 |
| enrolment | (208-353) | (203-314) | |
| Median (IQR) number of transplants | 1 | 1 | 0.59 |
| | (1-1) | (1-1) | |
| Median (IQR) serum creatinine at | 117 | 128 | 0.24 |
| enrolment | (92-165) | (108-159) | |
| Median (IQR) eGFR at enrolment | 51 | 44 | 0.16 |
| | (36-64) | (34-57) | |
| Immunosuppression at enrolment | | | |
| % on calcineurin inhibitor | 83% | 81% | 0.74 |
| % on azathiaprine | 78% | 65% | 0.12 |
| % on mycophenolate | 7% | 16% | 0.12 |
| % on sirolimus | 7% | 4% | 0.68 |
| % on steroids | 41% | 39% | 0.82 |
| Clinical phenotype | | | |
| % smoker (past or current) | 34% | 39% | 0.60 |
| % family history of malignancy | 53% | 35% | 0.04 |
| % personal history of non-NMSC | 9% | 19% | 0.16 |
| malignancy | | | |
| % reporting chronic UV exposure | 64% | 56% | 0.36 |
| Median (IQR) Harwood clinical risk score | 4 | 3 | 0.09 |
| | (2-4) | (2-4) | |
| Median (IQR) Urwin clinical risk score | 2 | 1 | 0.01 |
| | (1-3) | (0-3) | |
| Median (IQR) Harden clinical risk score | 5.3 | 4.9 | 0.38 |
| | (4.2-6.2) | (4.3-5.7) | |
| Immune phenotype | | | |
| Number of CD8+ CD57+ cells (per ul | 123 | 145 | 0.45 |
| blood) | (34-233) | (76-304) | |
| Median (IQR) % CD8+ expressing CD57 | 67 | 49 | 0.13 |
| | (41-76)% | (35-70)% | |

### Prediction of SCC Development Using Clinical Markers

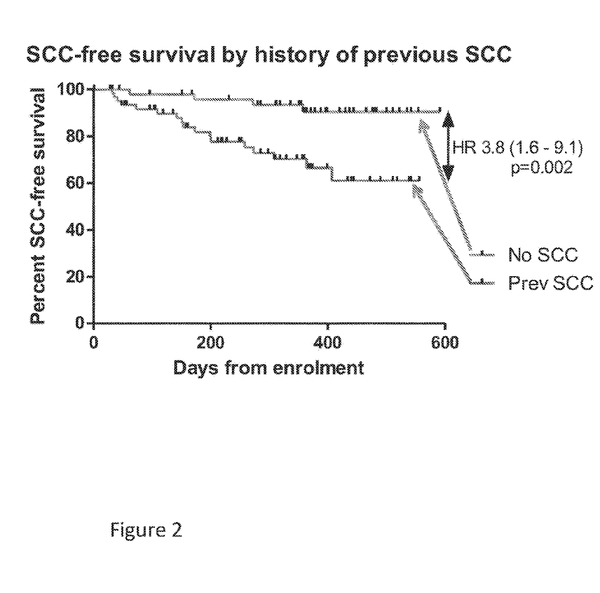

Participants in the study were followed up for a median (IQR) period of 371 (285-472) days, representing a total of 34890 days ‘at risk’ of SCC. During the follow up period 22 RTR developed a total of 36 SCC. RTR with a history of SCC were at nearly four-fold risk of further SCC, as demonstrated in FIG. 2.

A univariate analysis was performed to assess clinical factors predictive of further SCC development in this cohort and it was found that age at recruitment and at first transplantation, as well as history of previous SCC, were predictive of further SCC development, as detailed in Table 3. Gender, duration of immunosuppression, CMV serostatus, renal function and immunosuppression type were not predictive of SCC development (data not shown). Only the Urwin risk score was found to be predictive of further SCC development, though both the Harwood and Harden risk scores trended towards significance.

Using multivariate analysis to account for potential confounding between factors, it was observed that, correcting for age, all three risk scores lost predictive value. Furthermore, age at first transplant also lost predictive value, leaving age at enrolment and previous SCC as the only independently predictive clinical markers for SCC development.

TABLE 3
Regression analyses for SCC development during the study using clinical
markers. Multivariate analysis was performed using previous SCC, age at
transplantation and age at enrolment as covariates, whilst each individual risk score was
assessed using age as a covariate. For continuous variables, such as age, the hazard
ratio is per unit (e.g. year) increase.

| | Univariate analysis | | | |
|---|---|---|---|---|
| | Hazard Ratio (HR) | | Multivariate analysis* | |
| Variable | (95% CI) | p | HR (95% CI) | p |
| Increasing age at | 1.07 (1.02-1.11) | 0.002 | 1.05 (1.01-1.09) | 0.016 |
| enrolment | | | | |
| Increasing age at 1^{st} | 1.03 (1.00-1.07) | 0.050 | 0.99 (0.94-1.04) | 0.648 |
| transplant | | | | |
| Previous SCC | 5.48 (1.85-16.2) | 0.002 | 4.39 (1.45-13.3) | 0.009 |
| Chronic UV exposure | 2.75 (1.01-7.47) | 0.047 | 2.65 (0.96-7.36) | 0.06 |
| Clinical risk scores | | | | |
| ↑ Harden risk score | 1.31 (0.99-1.7) | 0.06 | 1.01 (0.71-1.46) | 0.94 |
| ↑ Urwin risk score | 1.50 (1.12-2.02) | 0.007 | 1.31 (0.95-1.80) | 0.10 |
| ↑ Harwood risk score | 1.43 (0.97-2.12) | 0.07 | 0.84 (0.47-1.50) | 0.55 |

### Prediction of SCC Using Percentage of CD8+ Cells Expressing CD57

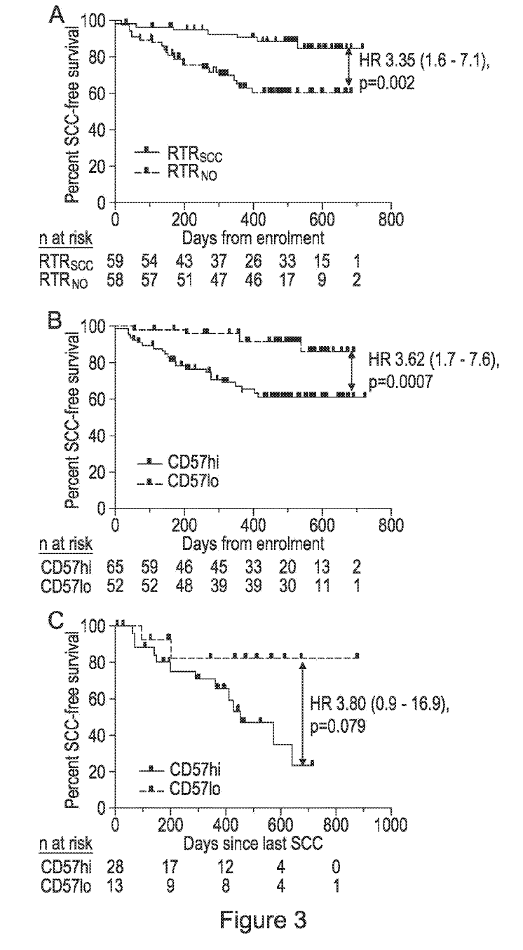

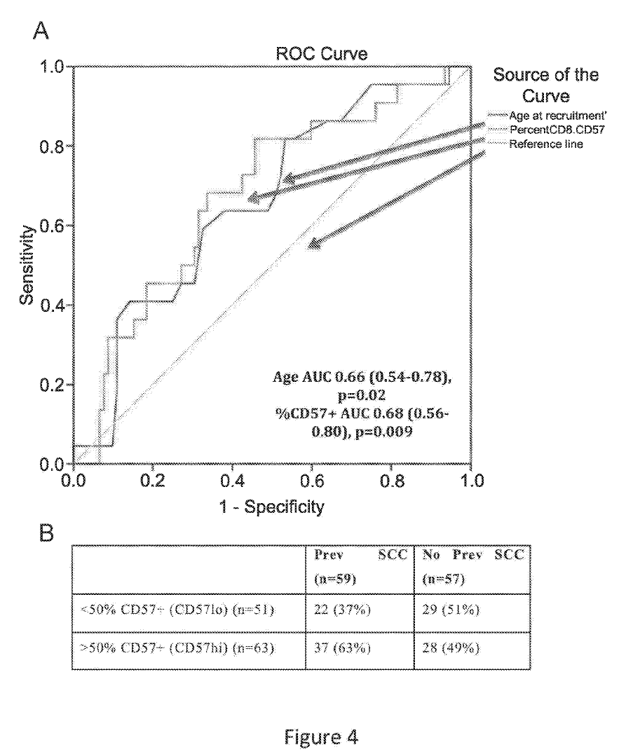

Having established the performance of clinical risk markers in this population, their performance was compared with the percentage of CD57+ cells as predictors of further SCC. A receiver operating characteristic (ROC) curve (FIG. 4) was produced. This gave a similar predictive performance of CD57 and age, as assessed by area under the curve. It was observed that CD57 percentage was bimodal (largely due to the influence of CMV serostatus) and which allow the RTR to be stratified into those with a majority (>50%) of CD8+ cells expressing CD57 (CD57^{hi}) and those with a minority)(CD57^{lo}. RTR with a history of SCC tended to be in the CD57hi group, though this did not reach significance (FIG. 3). ROC curves were also construced with regards to BCC development (n=13) and other malignancy (n=3), but this was not predictive.

Using this stratification, it was found on univariate analysis that RTR who were CD57hi were over four times more likely to develop SCC during follow-up than CD57lo RTR (Table 4). When corrected for age, only a history of previous SCC and being CD57hi remained predictive of the development of SCC. CD57hi status remained predictive of the development of SCC, independent of previous SCC and increasing age. Given the impact of CMV seropositivity on the proportion of CD57+ cells (CMV seropositive RTR were eighteen times more likely to be CD57hi), further investigation into this relationship was undertaken but it was found on multivariate analysis that CD57 remained independently predictive of SCC development (HR 4.4 (1.49-13.1), p=0.007) when adjusted for CMV serostatus.

TABLE 4
Regression analysis using CD57hi and CD57lo to stratify. All three variables
were used as covariates for multivariate analysis.

| | Univariate | | Multivariate | |
|---|---|---|---|---|
| Variable | HR (95% CI) | p | HR (95% CI) | p |
| ↑ age at recruitment | 1.07 | 0.002 | 1.03 | 0.14 |
| | (1.02-1.11) | | (0.99-1.08) | |
| Previous SCC | 5.48 | 0.002 | 4.9 | 0.005 |
| | (1.85-16.2) | | (1.63-14.5) | |
| >50% CD8 expressing CD57 | 4.45 | 0.002 | 3.9 | 0.015 |
| (CD57hi) | (1.50-13.2) | | (1.30-11.5) | |

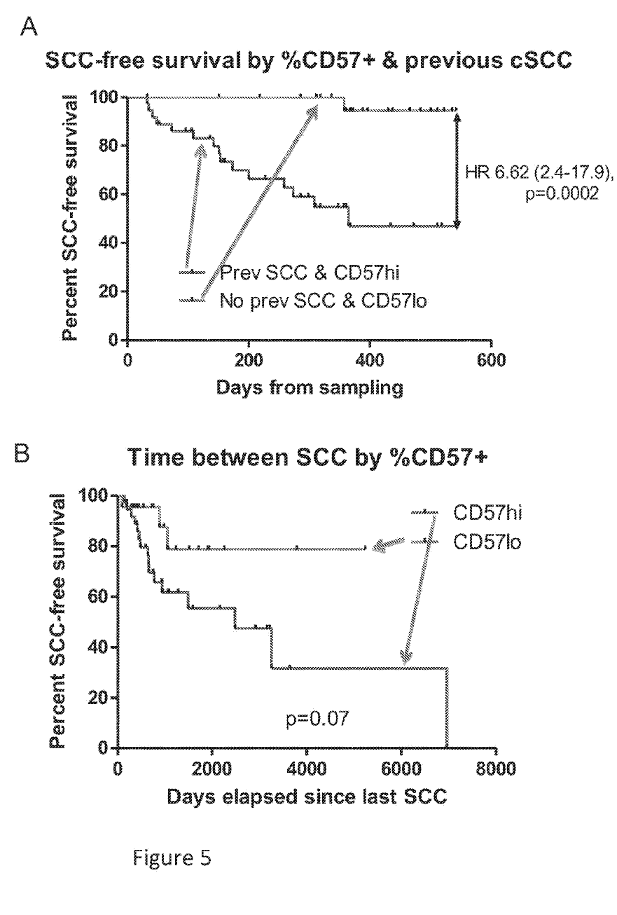

RTR who were highest risk (previous SCC and CD57hi) were nearly seven-fold more likely to develop an SCC during study follow-up compared to those who were lowest risk (FIG. 5A). Furthermore, when time since previous SCC was examined in those who were CD57hi compared to those who were CD57lo, it was observed that those who were CD57hi trended towards earlier SCC recurrence (FIG. 5B), and were three-fold more likely to develop an SCC during study follow-up (HR 3.01 (1.17-7.73), p=0.02, data not shown).

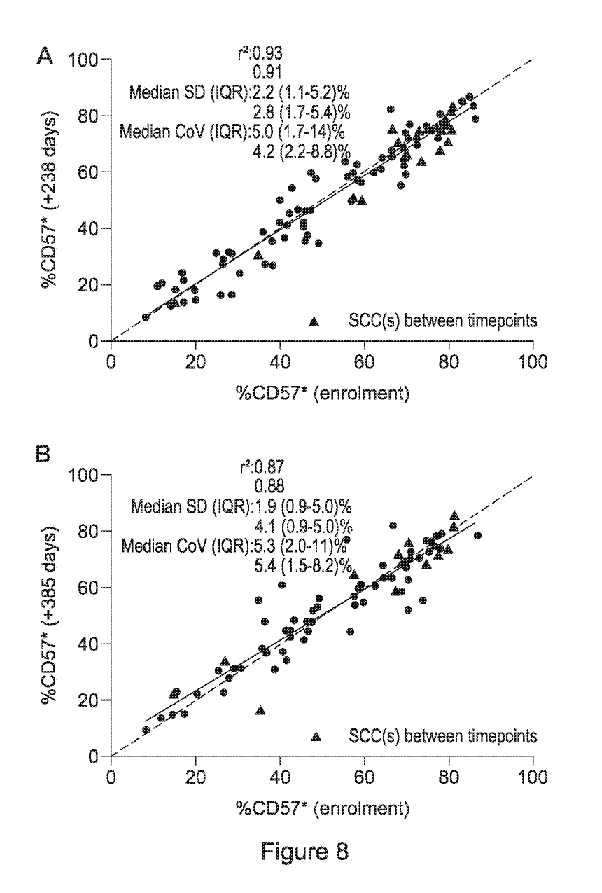

The data presented in FIG. 8 demonstrates that CD57 is stable for over a 1-year period and with SCC development, further supporting its usefulness as a marker.

### Immunological Features of CD8+CD57+ Cells

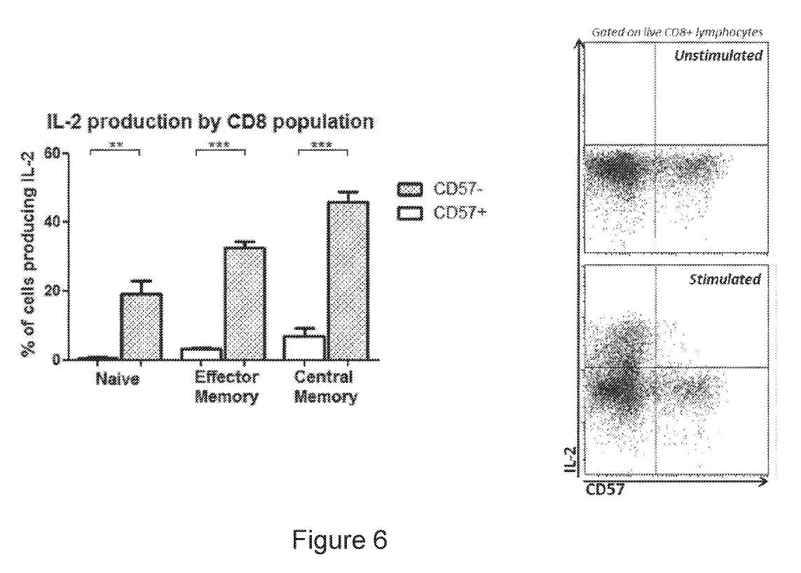

CD8+CD57+ cells were further analysed ex vivo. These cells were predominantly derived from effector memory populations, and were markedly impaired in the production of IL-2 in response to polyclonal stimulation in healthy individuals (FIG. 6). Furthermore, an increased proportion of CD8+CD57+ cells was associated with a reduction in the number of naïve and central memory CD8 T-cells. The percentage of CD8+CD57+ cells correlates with the percentage of CD4+CD57+ cells, which are also impaired in the ability to produce IL-2.

### CONCLUSIONS & CONTEXT

This study is the first to directly compare the performance of clinical risk markers against immunological risk markers in the prediction of SCC in long-term RTR, who can generally be considered to be at globally ‘high risk’ due to cumulative time of immunosuppression and increased age. The proportion of CD57-expressing CD8+ T-cells represents the first immunological marker that has been demonstrated to have superior predictive power beyond traditionally utilised clinical indicators, in this setting.

The identification of a marker that allows stratification of RTR with regards to risk of SCC has a number of implications. Firstly, this may allow for more intensive screening in those who are high risk, whilst those at lower risk may require less intensive follow-up. This potentially could free dermatological resources to monitor and intervene with those at highest risk of SCC. Secondly, CD8+CD57+ cells are thought to represent an ‘exhausted’ phenotype; data supported by the findings presented here. It may well be that the immune system's impaired response to the development of malignancy is indicative of an impaired response to the graft itself, which may underlie the ability to frequently (though not invariably) reduce immunosuppression in these patients without the development of graft dysfunction. Stratification by peripheral blood CD57 proportion may act as a biomarker to identify those RTR that may be able to reduce their immunosuppression prior to the development of malignancy. An additional benefit is a financial one: standard double therapy immunosuppression (i.e. ciclosporin and azathioprine) costs in the region of £1800 per annum per patient (A. Devaney, personal communication). A reduction in dosage of these therapies in a subset of RTR may have ongoing cost-saving implications for the healthcare provider.

CD57 has been shown to be a poor prognostic marker in a number of malignancies, including gastric, melanoma and renal cell. However, these studies looked only at participants who had malignancy at the time of sampling. Thus these studies did not identify CD57 in the context of predicting de novo malignancy development, but rather progression of established malignancy.

CD57 has been previously investigated in the setting of both transplantation and malignancy. Boleslawski (2011) looked at a cohort of liver transplant recipients and found that a decreased proportion of CD8+CD28+(and by inversion an increased proportion of CD8+CD28−) cells predicted the development of malignancy in the first ten years post-transplant[32]. CD8+CD57+ cells are often (though not always) CD28-negative (a finding confirmed by the inventors with a strong correlation between these two populations). Liver transplantation generally requires much lower doses of immunosuppression as tolerance is a more common phenomenon (up to 30% of liver transplant recipients are able to cease immunosuppression) in comparison to kidney transplantation (where tolerance is thought to be very rare) and thus the types of malignancy encountered post-transplant are likely to differ, with a relative overrepresentation of liver cancer (which isn't seen in renal transplantation). Only a third of the malignancies encountered during follow-up in the Boleslawski study were skin malignancies, and these were experienced by only 6% of the study cohort, highlighting that skin malignancy is generally encountered much later in the post-transplant course. Courivaud analysed malignancy in the first decade following renal transplantation and found that CMV seropositive RTR were more likely to develop malignancy than those who were CMV seronegative. In addition, this study found that CMV seropositivity was associated with an increased percentage of CD8+CD57+IL-2− cells; however, they did not use CD57 itself as a predictive marker for malignancy. The inventors also have observed that CMV seropositivity is associated with an eighteen-fold increased likelihood of being CD57hi, but CMV seropositivity itself was not predictive of SCC development, whilst the proportion of CD57+ cells remained independently predictive. Given the data above, it is likely that CD57 is predictive of the development of other malignancies in long-term RTR, but the paucity of these events in this study means we are unable to formally assess this.

The poor performance of clinical risk scores may be due to a number of reasons. The Harden clinical risk score was developed in a cohort of RTR who were in the first 10 years post-transplant, where skin cancer is relatively underrepresented. Secondly, the populations in this study may differ from those where the measures were developed. The Urwin risk score was developed utilising an Australian population, and so gives relative prominence to features such as duration of time in a tropical climate and pre-transplant NMSC, both of which are unusual in a British cohort.

### REFERENCES

1. Survival Rates Following Transplantation. [cited 2014 16 Apr.]; Available from: www.organdonation.nhs.uk/ukt/statistics/transplant_activity_report/current_activity_reports/ukt/survival_rates_following_transplantation.pdf.
2. Matas, A. J., et al., OPTN/SRTR 2011 Annual Data Report: kidney. Am J Transplant, 2013. 13 Suppl 1: p. 11-46.
3. Pilmore, H., et al., Reduction in cardiovascular death after kidney transplantation. Transplantation, 2010. 89(7): p. 851-7.
4. Euvrard, S., et al., Subsequent skin cancers in kidney and heart transplant recipients after the first squamous cell carcinoma. Transplantation, 2006. 81(8): p. 1093-100.
5. Baker, R., A. Jardine, and P. Andrews. Post-operative Care of the Kidney Transplant Recipient. 2011 5 Feb. 2011 [cited 2014 17 Oct.]; Available from: http://www.renal.org/guidelines/modules/post-operative-care-of-the-kidney-transplant-recipient#sthash.OIcp7tZp.dpbs.
6. US Renal Data System. USRDS 2012 Annual Data Report: Atlas of End-Stage Renal Disease in the United States. 2012, National Institutes of Health, National Institute of Diabetes and Digestive and Kidney Diseases, Bethesda, Md.
7. Harden, P. N., et al., Annual incidence and predicted risk of nonmelanoma skin cancer in renal transplant recipients. Transplant Proc, 2001. 33(1-2): p. 1302-4.
8. Harwood, C. A., et al., A Surveillance Model for Skin Cancer in Organ Transplant Recipients: A 22-Year Prospective Study in an Ethnically Diverse Population. Am J Transplant, 2012.
9. Urwin, H. R., et al., Predicting risk of nonmelanoma skin cancer and premalignant skin lesions in renal transplant recipients. Transplantation, 2009. 87(11): p. 1667-71.
10. Dantal, J., et al., Effect of long-term immunosuppression in kidney-graft recipients on cancer incidence: randomised comparison of two cyclosporin regimens. Lancet, 1998. 351(9103): p. 623-8.
11. Carroll, R. P., et al., Immune phenotype predicts risk for posttransplantation squamous cell carcinoma. J Am Soc Nephrol, 2010. 21(4): p. 713-22.
12. Hope, C. M., et al., The immune phenotype may relate to cancer development in kidney transplant recipients. Kidney Int, 2014.
13. Sherston, S. N., et al., Demethylation of the TSDR Is a Marker of Squamous Cell Carcinoma in Transplant Recipients. Am J Transplant, 2014.
14. Kosmidis, M., et al., Immunosuppression affects CD4+ mRNA expression and induces Th2 dominance in the microenvironment of cutaneous squamous cell carcinoma in organ transplant recipients. J Immunother, 2010. 33(5): p. 538-46.
15. Muhleisen, B., et al., Progression of cutaneous squamous cell carcinoma in immunosuppressed patients is associated with reduced CD123+ and FOXP3+ cells in the perineoplastic inflammatory infiltrate. Histopathology, 2009. 55(1): p. 67-76.
16. Zhang, S., et al., Increased Tc22 and Treg/CD8 ratio contribute to aggressive growth of transplant associated squamous cell carcinoma. PLoS One, 2013. 8(5): p. e62154.
17. Strioga, M., V. Pasukoniene, and D. Characiejus, CD8+ CD28− and CD8+ CD57+ T cells and their role in health and disease. Immunology, 2011. 134(1): p. 17-32.
18. Cebo, C., et al., Function and molecular modeling of the interaction between human interleukin 6 and its HNK-1 oligosaccharide ligands. J Biol Chem, 2002. 277(14): p. 12246-52.
19. Nielsen, C. M., et al., Functional Significance of CD57 Expression on Human NK Cells and Relevance to Disease. Front Immunol, 2013. 4: p. 422.
20. Brenchley, J. M., et al., Expression of CD57 defines replicative senescence and antigen-induced apoptotic death of CD8+ T cells. Blood, 2003. 101(7): p. 2711-20.
21. Chong, L. K., et al., Proliferation and interleukin 5 production by CD8hi CD57+ T cells. Eur J Immunol, 2008. 38(4): p. 995-1000.
22. Akagi, J. and H. Baba, Prognostic value of CD57(+) T lymphocytes in the peripheral blood of patients with advanced gastric cancer. Int J Clin Oncol, 2008. 13(6): p. 528-35.
23. Characiejus, D., et al., Peripheral blood CD8highCD57+ lymphocyte levels may predict outcome in melanoma patients treated with adjuvant interferon-alpha. Anticancer Res, 2008. 28(2B): p. 1139-42.
24. Characiejus, D., et al., Predictive value of CD8highCD57+ lymphocyte subset in interferon therapy of patients with renal cell carcinoma. Anticancer Res, 2002. 22(6B): p. 3679-83.
25. Filaci, G., et al., CD8+ CD28− T regulatory lymphocytes inhibiting T cell proliferative and cytotoxic functions infiltrate human cancers. J Immunol, 2007. 179(7): p. 4323-34.
26. Focosi, D., et al., CD57+ T lymphocytes and functional immune deficiency. J Leukoc Biol, 2010. 87(1): p. 107-16.
27. Hadrup, S. R., et al., Longitudinal studies of clonally expanded CD8 T cells reveal a repertoire shrinkage predicting mortality and an increased number of dysfunctional cytomegalovirus-specific T cells in the very elderly. J Immunol, 2006. 176(4): p. 2645-53.
28. Hebib, C., et al., Pattern of cytokine expression in circulation CD57+ T cells from long-term renal allograft recipients. Transpl Immunol, 1998. 6(1): p. 39-47.
29. Le Priol, Y., et al., High cytotoxic and specific migratory potencies of senescent CD8+ CD57+ cells in HIV-infected and uninfected individuals. J Immunol, 2006. 177(8): p. 5145-54.
30. Sadat-Sowti, B., et al., An inhibitor of cytotoxic functions produced by CD8+CD57+ T lymphocytes from patients suffering from AIDS and immunosuppressed bone marrow recipients. Eur J Immunol, 1994. 24(11): p. 2882-8.
31. Wang, E. C., et al., CD8high+ (CD57+) T cells in patients with rheumatoid arthritis. Arthritis Rheum, 1997. 40(2): p. 237-48.
32. Boleslawski, E., et al., CD28 expression by peripheral blood lymphocytes as a potential predictor of the development of de novo malignancies in long-term survivors after liver transplantation. Liver Transpl, 2011. 17(3): p. 299-305.

1. A method of determining an increased risk of cancer in an immunosuppressed patient, the method comprising:
(a) determining the percentage of CD8+CD57+ T-cells in a population of CD8+ T-cells in a sample from the patient;
wherein a percentage of 40% or greater of CD8+CD57+ T-cells is indicative of an increased risk of cancer; and/or
(b) determining the percentage of CD4+CD57+ T-cells in a population of CD4+ T-cells in a sample from the patient;
wherein a percentage of 10% or greater of CD4+CD57+ T-cells is indicative of an increased risk of cancer.

2. The method according to claim 1, wherein a percentage of 50% or greater of CD8+CD57+ T-cells is indicative of an increased risk of cancer.

3. The method according to any preceding claim, further comprising the step of selecting patients determined to be at increased risk of cancer for one or more of:
increased surveillance for cancer;
modifying the immunosuppressive therapy regime; or
providing preventative therapy for cancer, and optionally wherein the preventative therapy for cancer is anti-cancer therapy.

4. The method according to any preceding claim, further comprising determining the percentage of CD8+CD28− T-cells in the population of CD8+ T-cells, wherein a percentage of 40% or greater of CD28− T-cells is indicative of an increased risk of cancer.

5. A method of determining an increased risk of cancer in an immunosuppressed patient, the method comprising:
determining the percentage of CD8+CD28− T-cells in a sample of CD8+ T-cells from the patient;
wherein a percentage of 40% or greater is indicative of an increased risk of cancer.

6. The method according to any preceding claim, wherein the immunosuppression is due to the patient receiving immunosuppressive therapy.

7. The method according to claim 6, wherein the immunosuppressive therapy is following a transplant.

8. The method according to claim 7, wherein the transplant comprises a kidney transplant.

9. The method according to claim 7, wherein the transplant comprises a heart and/or lung transplant.

10. The method according to any of claims 7 to 9, wherein the transplant is not a liver transplant.

11. The method according to any of claims 6 to 9, wherein the immunosuppressive therapy does not comprise a regular and ongoing dosage of steroid.

12. The method according to any preceding claim, comprising the step of selecting patients determined to be at increased risk of cancer for increased surveillance for cancer.

13. The method according to any preceding claim, comprising the step of selecting patients determined to be at increased risk of cancer for modifying the immunosuppressive therapy regime of the patient.

14. The method according to any preceding claim, comprising the step of selecting patients determined to be at increased risk of cancer for providing preventative therapy for cancer.

15. The method according to any of claims 12 to 14, wherein for selected patients determined to be at increased risk of cancer the method further comprises one or more steps comprising:
increasing surveillance for cancer;
modifying the immunosuppressive therapy regime; or
providing preventative therapy for cancer.

16. The method according to any of claims 13 to 15, wherein modifying the immunosuppressive therapy regime comprises one or more of:
reduction of dose and/or frequency of immunosuppressive therapy;
switching one or more immunosuppressive drugs to an alternative immunosuppressive drug(s);
reducing the number of different immunosuppressive drugs administered to the patient.

17. The method according to any of claims 13 to 16, wherein modifying the immunosuppressive therapy regime comprises reduction of dose of immunosuppressive therapy by at least 10% reduction.

18. The method according to any of claims 13 to 17, wherein modifying the immunosuppressive therapy regime comprises reduction of frequency of immunosuppressive therapy.

19. The method according to any of claims 13 to 18, wherein modifying the immunosuppressive therapy regime comprises switching one or more immunosuppressive drugs to an alternative immunosuppressive drug(s).

20. The method according to any of claims 13 to 19, wherein modifying the immunosuppressive therapy regime comprises reducing the number of different immunosuppressive drugs administered to the patient.

21. The method according to any of claims 13 to 20, wherein modification of the immunosuppressive therapy regime is accompanied by an increase in surveillance of transplant rejection.

22. The method according to any preceding claim, wherein the percentage of 40% or greater may be indicative of a risk of cancer after a period of at least 5 years following the onset of immunosuppression in the patient.

23. The method according to any preceding claim, wherein the cancer is skin cancer.

24. The method according to claim 23, wherein the skin cancer is SCC (squamous cell carcinoma).

25. Use of CD57 as a biomarker for determining an increased risk of cancer in a patient receiving immunosuppressive therapy.

26. Use of CD28 as a biomarker for determining an increased risk of cancer in a patient receiving immunosuppressive therapy following a kidney transplant; optionally wherein the cancer is SCC.

27. A kit for determining an increased risk of cancer in a patient receiving immunosuppressive therapy comprising:
a CD8 binding agent
a CD57 binding agent and/or a CD28 binding agent.

28. A kit for determining an increased risk of cancer in a patient receiving immunosuppressive therapy comprising:
a CD4 binding agent
a CD57 binding agent and/or a CD28 binding agent.

29. A method, use or kit as substantially described herein, optionally with reference to the accompanying figures.
